(19) United States
(12) Patent Application Publication
(10) Pub. No.: US 2024/0009670 A1
(43) Pub. Date: Jan. 11, 2024

(54) ASSAY DEVICE EMPLOYING FLUORESCENT LABELS

(71) Applicant: ELLUME LIMITED, East Brisbane, Queensland (AU)

(72) Inventors: Romulo DE MACEDO, East Brisbane, Queensland (AU); Geoff BYWATER, East Brisbane, Queensland (AU)

(21) Appl. No.: 18/252,702

(22) PCT Filed: Nov. 12, 2021
(86) PCT No.: PCT/AU2021/051342

§ 371 Date: May 11, 2023

(30) Foreign Application Priority Data
Nov. 12, 2020 (AU) 2020267255

(51) Int. Cl. B01L 3/00; G01N 33/543; G01N 21/64

(52) U.S. Cl. CPC B01L 3/502761; G01N 33/54386; G01N 21/6428; B01L 2200/16; B01L 2200/0652; B01L 2300/168; B01L 2300/0654; G01N 2021/6439

(57) ABSTRACT

An assay device is disclosed comprising a housing and a test portion, electronic circuitry and a one-piece optical component each a least partially located in the housing. The test portion comprises one or more test zones adapted to receive an analyte and a fluorescent label associated with the analyte, the fluorescent label being excitable by excitation light and adapted to emit emission light upon excitation by excitation light. The electronic circuitry comprises one or more light sources and one or more light detectors. The optical component comprises one or more excitation light guides adapted to guide excitation light from the one or more light sources to the one or more test zones, and one or more emission light guides adapted to guide emission light from the one or more test zone to the one or more light detectors.

### CROSS REFERENCE TO RELATED CASES

This application claims the benefit of priority from Australian Patent Application No. 2020267255, filed on 12 Nov. 2020, which is incorporated by reference herein, in its entirety.

### TECHNICAL FIELD

The field of the present disclosure relates to devices and methods for determination of the presence, absence or amount of a biological entity in a human or animal body.

### BACKGROUND

There exist many types of diagnostic devices for identifying target analytes and therefore target medical conditions in a person or animal. Increasingly, these devices are being designed for home use. The devices analyse a biological sample provided by the person or animal, such as a urine sample, blood sample or otherwise, and identify an analyte in the sample that provides a marker for the target condition.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY

According to a first aspect, the present disclosure provides an assay device comprising:
a housing; and
a test portion, electronic circuitry and an optical component, each a least partially located in the housing; wherein
the test portion comprises one or more test zones adapted to receive an analyte and a fluorescent label associated with the analyte, the fluorescent label being excitable by excitation light and adapted to emit emission light upon excitation by excitation light;
the electronic circuitry comprises one or more light sources and one or more light detectors; and
the optical component is a one-piece component that comprises one or more excitation light guides adapted to guide excitation light from the one or more light sources to the one or more test zones, and one or more emission light guides adapted to guide emission light from the one or more test zones to the one or more light detectors.

The optical component may be one-piece by being moulded, cast or otherwise formed as single piece that includes the one or more excitation light guides and the one or more emission light guides. For example, the optical component may be injection moulded as a single piece. The optical component may be plastic. The optical component may be considered monolithic, solid and unbroken.

The excitation and emission light guides may perform multiple functions. In addition to guiding light to and from the test zones, the light guides may also focus, collimate and/or diverge light, for example.

The one or more excitation light guides may be adapted to guide (transmit) excitation light that has a wavelength in an excitation wavelength band and the one or more emission light guides may be adapted to guide (transmit) emission light that has a wavelength in an emission wavelength band, the emission wavelength band being different from the excitation wavelength band.

The optical component may have substantially no spectral filtering function. For example, the optical component may include no spectral filtering material (e.g. dyes or otherwise) to block any light that has wavelength outside of the excitation wavelength band and no spectral filtering material (e.g. dyes or otherwise) to block any light that has a wavelength outside of the emission wavelength band. Since it may have substantially no spectral filtering function, the optical component may formed of a substantially transparent, clear material. Additionally or alternatively, the optical component may be formed of homogenous material since it may not need to include any spectral filtering materials that are different for the one or more excitation light guides of the optical component in comparison to the one or more emission light guides of the optical component.

Nevertheless, the assay device may include one or more spectral filters that are separate from the optical component. For example, the assay device may include one or more spectral filters associated or integrated with the one or more light detectors. For example, one or more light detectors such as photodetectors or phototransistors may be provided that include a spectral filter. The spectral filter may be a filter, such as a long pass filter, that blocks any light that (a) has a wavelength outside of the emission wavelength band and/or (b) has a wavelength inside the excitation wavelength band, for example.

To achieve this spectral filtering, light transmitting material may be used that is impregnated with a filtering material such as a spectral filtering dye. A single dye or a mixture of dyes (a compound dye) may be employed to achieve desired spectral filtering properties.

In general, the excitation wavelength band may have a relatively low wavelength and the emission wavelength band may have a relatively high wavelength. At a general level, providing relatively large differences between central wavelengths of the transmission bands of the different light guides can ensure that higher discrimination between the excitation light and the emission light is possible during spectral filtering. Most notably, it can allow a significant reduction in the amount of excitation light that could be incident on the relevant light detector, including through use of long pass spectral filtering at the light detector. It may ensure that, to the greatest extent possible, the only light that is incident on or sensed by the light detector is light emitted from fluorescent labels at the test zone.

By employing fluorescent labels, sensitivity gains may be achieved over more commonly deployed labels in assays, such as gold nanoparticles (colloidal gold). Further, sensitivity gains can be achieved by employing a fluorescent label that exhibits a relatively large Stokes shift. For example, the excitation wavelength band and the emission wavelength band may have central or peak wavelengths that are at least 200 nm apart, or at least 250 nm apart, or at least 300 nm apart, or at least 350 nm apart, at least 400 nm apart, at least 450 nm apart or otherwise. For example, the excitation wavelength band may have a peak wavelength between about 325 and 500 nm or between about 375 nm and 430 nm and the emission wavelength band may have a peak wavelength between about 650 and 1200 nm or between about 700 and 1100 nm. In one embodiment, the excitation wavelength band has a peak wavelength of about 405 nm and the emission wavelength band has a peak wavelength of about 900 nm. An example of a flurophore that can exhibit a relatively large Stokes shift (e.g., a shift of greater than 250 nm, or greater than 300 nm, or greater than 350 nm, or greater than 400 nm, or greater than 450 nm) is a quantum dot. In general, therefore, quantum dots can be used as fluorescent labels in devices of the present disclosure. However, other types of fluorescent labels may be used.

It has been found that quantum dots with higher peak excitation wavelengths generally exhibit larger Stokes shifts. Quantum dots that exhibit relatively large Stokes shifts may have peak emission wavelengths of e.g., greater than 600 nm, greater than 650 nm, greater than 700 nm, or greater than 750 nm, or about 800 nm, for example. By employing fluorescent labels with relatively large Stoke shifts, and/or with relatively high peak emission wavelengths, problems associated with autofluorescence may be minimised.

Autofluorescence may occur within a variety of substances within an assay device, such as the substrate of a test strip and backing layers, etc. Typically autofluorescence occurs with excitation and emission wavelength levels below about 650 nm. Therefore, by employing fluorescent labels that exhibit relatively large Stokes shifts and/or fluoresce above e.g., 650 nm or greater, separation/filtering of any autofluorescent emission light from the fluorescent label emission light (i.e. from the emission light of interest) can be more straightforward to achieve. Furthermore, when the peak wavelength is e.g., above about 650 nm, the filtering may need only block wavelengths below the peak wavelength of the fluorescent label, since autofluorescence may substantially occur only at wavelengths below the peak wavelength of the fluorescent label. In contrast, if a fluorescent label such as Europium is employed, for example, which has a peak emission wavelength of about 615 nm, filtering of autofluorescence light can pose more considerable problems.

An alternative method of minimising the effects of autofluorescence that may be employed is Time Resolved Fluorescence. However, while this is effective at reducing the autofluorescence background, it has several major disadvantages relating to, for example, the complexity of the electronics needed to carry out the technique and the ability to integrate signals over time to any great degree.

In any of the aspects disclosed herein, to further reduce the amount of excitation light, or other non-emission light, incident on the one or more light detectors, the assay device may comprise one or more light baffles. The light baffles may act to shield light from different parts of the assay device. The assay device may comprise a housing and the light baffles may form part of the housing. The light baffles may be located between the light sources and the light detectors, and/or between the excitation and emission light guides.

The one or more excitation light guides may comprise a light collimator lens adjacent at least one of the light sources, in order to collimate light arriving from the light source into the excitation light guide. The one or more excitation light guides may also comprise refractive and/or reflective surfaces. The one or more excitation light guides may comprise a light exit face. The one or more test zones may be located on a substrate, e.g., a lateral flow test strip, and the light exit face may extend across a plane that is substantially perpendicular to a plane of the substrate on which the test zones are located. At least one refractive and/or reflective surface may be provided at a substantially opposite side of the light guide to the light exit face. The at least one refractive and/or reflective surface may be curved. A best fit plane extending through the curved surface may be at an angle from the plane of the light exit face. The angle may be between about 20 and 70 degrees, or between 30 and 45 degrees or otherwise. The curved surface may extend substantially an entire length of the light guide in a direction between electronic circuitry and the test portion. In general, the excitation light guides, including the refractive and/or reflective surfaces, may route excitation light efficiently between the light sources and the test zones. The refractive and/or reflective surfaces may act as a combined folding mirror and lens, providing optical power to the excitation light.

The one or more emission light guides may include curved refractive surfaces at one or both ends of the light guides. For example, at one or both ends of the light guides, a lens may be provided, e.g., a ball lens, a half ball lens or a plano-convex lens. A spacer may be provided between the lenses. The spacer may be a cylindrical spacer. The spacer may provide a total-internal-reflection concentrator between the two lenses.

The one or more test zones may comprise at least first and second test zones. The optical component may comprise at least first and second excitation light guides and first and second emission light guides. The first excitation light guide may be adapted to guide light from the one or more light sources to the first test zone and the first emission light guide may adapted to guide light from the first test zone to the one or more light detectors. The second excitation light guide may be adapted to guide light from the one or more light sources to the second test zone and the second emission light guide may be adapted to guide light from the second test zone to the one or more light detectors.

By providing at least two test zones, the device may be used to test for the presence of different target analytes in a biological sample. For example, the first test zone may be adapted to receive a first target analyte and the second test zone may be adapted to receive a second target analyte. In one embodiment, the first target analyte may be influenza A (e.g., a nucleoprotein of influenza A) and the second target analyte may be influenza B (e.g., a nucleoprotein of influenza B). Nevertheless, a variety of different analytes may be tested using the device according to the present disclosure.

While providing at least two test zones may allow for testing of the presence of different target analytes, as an alternative approach, one of the test zones may be provided as a control. Where target analytes such as influenza A and influenza B are to be detected, since the presence of one of these analytes in a body is generally understood to be mutually exclusive of the presence of the other of these analytes in the body, one of the two test zones can be used to perform a control function. For example, background fluorescence or autofluorescence can be deduced from the test zone that does not have a fluorescently labelled target analyte bound thereto, and the value of this fluorescence can be considered when determining a degree of fluorescence attributable to the presence of fluorescently labelled target analyte at the other of the test zones.

In general, the electronic circuitry and optical component can form part of an electronic reader. The electronic circuitry may include a processor and a non-transitory computer-readable memory medium, the non-transitory computer-readable memory medium comprising instructions that cause the processor to carry out steps to monitor the one or more target analytes, which may be present in a biological sample, including to determine the presence, absence or amount of one or more analytes of interest in the biological sample and/or to determine a biological condition based on the presence, absence or amount of the one or more target analytes in the biological sample.

The test portion may be a lateral flow test strip or another device, element or assembly that provides a change in fluorescence at an illuminated test zone when a target analyte is determined to be present. The fluorescence may provide a form of signal at the test zone that is monitored by the reader.

In one embodiment, the assay device may make a determination about at least a first target analyte (first analyte of interest) in a biological sample by:
monitoring levels of first and second signals at the first and second test zones over an assay period wherein, if the first analyte of interest is present in the sample, the first analyte is fluorescently labelled and wherein the presence of the fluorescently labelled first analyte in the sample causes the level of one of the first and second signals to increase during the assay period; and
monitoring a change between the first and second signal levels over a period of time during the assay period.

Additionally, the assay device may make a determination about a second target analyte (second analyte of interest) in the sample. If the second analyte of interest is present in the sample, the second analyte may be fluorescently labelled in the sample. The monitoring of the levels of first and second signals at the first and second test zones over an assay period may recognise that, if labelled first analyte is present in the sample, the level of one of the first and second signals may increase during the assay period and, if labelled second analyte is present in the sample, the level of the other one of the first and second signals may increase during the assay period. The presence of the second analyte of interest in the sample may be mutually exclusive of the presence of the first analyte of interest in the sample. The first analyte of interest may be an Influenza A analyte and second analyte of interest may an Influenza B analyte, or vice versa, for example.

The test portion may employ various conventional lateral flow techniques, which may rely on the forming of a sandwich assay, for example. Prior to being received at the first and second test zones, the sample may be combined with a first mobilisable capture reagent that is able to bind specifically to the first analyte of interest, if present in the sample, to form a plurality of fluorescently-labelled complexes. One of the first and second test zones may comprise an immobilised capture reagent being able to bind specifically to the fluorescently-labelled complexes to immobilize the fluorescently-labelled complexes. On the other hand, the other of the first and second test zones may be configured so that it does not immobilize or has a reduced ability to immobilize a plurality of the fluorescently-labelled complexes. Thus, when the analyte of interest is present in the sample, the fluorescently-labelled complexes may accumulate at one of the first and second test zones over the assay period, and not the other.

In some embodiments, fluorescent labelling of the analyte(s) of interest may occur separately to the lateral flow process. The labelling may occur upstream of the lateral flow process, e.g., as part of an incubation process of otherwise. The sample may be prepared in a solute form. Any labelled complexes may be distributed relatively uniformly throughout the sample. A relatively homogenous labelled sample may therefore be received at the first and second test zones. This may provide, during the assay period, a substantially linear increase of the signal level at one of the test zones and a substantially constant signal level at the other one of the test zones, for example.

By monitoring a change between the first and second signal levels over a period of time during the assay period, an accurate determination about the analyte of interest may be made through changes in relative measurements of signal levels. This monitoring approach may reduce any need for spectral filtering of both emission light and excitation light and may further support use of e.g. a long pass filter only, associated or integrated with the one or more light detectors, and/or use of the one-piece optical component that may include substantially no spectral filtering function.

A single light source may provide excitation light for both the first and second test zones. Alternatively, a plurality of light sources may be provided. For example, the one or more light sources may comprise at least first and second light sources, wherein the excitation light guided by the first excitation light guide is from the first light source and the excitation light guided by the second excitation light guide is from the second light source.

The first and second emission light guides may guide emission light from the first and second test zones to the same light detector. Thus, a light detector may be shared between both the portion of the optical path associated with the first test zone and the portion of the optical path associated with the second test zone. By using a single light detector, the size of the device and the manufacturing costs can be reduced. So that a single light detector can be used that can differentiate between light emitted from the first and second test zones, the first and second light sources may be ‘polled’ in turn. In other words, the first and second light sources may be turned substantially on or off at different times such that a time division multiplexed signal is effectively received by the light detector. As one example alternative approach, the device may be configured such that light of different frequencies is emitted from the test zones (e.g., by employing labels with different fluorescent properties adapted to be received at the different test zones). The light detector may be a frequency dependent photodetector that can differentiate between the signal strengths of light of different frequencies. To this end, a wavelength division multiplexed signal may effectively be received by the light detector.

Nonetheless, the assay device may comprise more than one light detector. For example, first and second light detectors may be provided that are adapted to receive emission light from the first and second test zones respectively.

Particularly, although not necessarily exclusively, when a single light detector is provided that is adapted to receive emission light from both the first and second test zones, the light detector may be located between the first and second light sources. Thus, the first and second light sources may be located on opposite sides of the at least one light detector. The optical component can direct light centrally from the light sources toward the at least one light detector.

The assay device may be a hand-held device. The device may differ in this regard from apparatus employed in a laboratory environment. The assay device may be a rapid diagnosis point-of-care test device, permitting testing in less than one hour, less than 30 minutes, less than 10 minutes, less than 5 minutes, or less than 2 minutes, for example. The device may be disposable, configured for single-use only. The device may be provided in sterile packaging prior to use. The device may provide a means for entirely non-invasive testing. The device may be used for testing in the veterinary field as well as in the field of human medicine.

The device may comprise a display, and the electronic circuitry, e.g., a processor of the electronic circuitry, may be connected to the display such that results of testing can be presented on the display.

In the present disclosure, the test portion may be fixed relative to the electronic circuit and the optical component. In this regard, the assay device may be provide for a ‘fixed optics’ solution to analyse the test portion. The position of the electronic circuitry, optical component and test portions may be fixed during the manufacturing process and prior to receipt of a biological sample on the assay device. The device may differ in this regard from apparatus in which the test portion is moved relative to an electronic reader, and/or from apparatus in which a test portion is inserted into a reader after receipt of a sample for testing. In one embodiment, the test portion of the present disclosure may comprise a registration hole adapted to receive a protuberance that may extend, for example, from the housing. By locating the protuberance in the registration hole, the test portion may be positioned in appropriate fixed relationship relative to the other components of the device. Furthermore, by providing a registration hole, during manufacture of the test portion, the hole may be used to align the strip with equipment that forms the test zones on the test portion.

The device may comprise chambers separated by the light baffles, e.g., a first chamber and a second chamber separated by a first light baffle, and a third chamber separated from the second chamber by a second light baffle. The first light source may be configured to emit light into the first chamber and the second light source may be configured to emit light into the third chamber. The first excitation light guide may be located in the first chamber and the second excitation light guide may be located in the third chamber. The one or more light detectors may be configured to receive light from the second chamber. Both the first and second emission light guides may be located in the second chamber. Whether separated by light baffles or otherwise, the excitation light guides and the emission light guides of the one-piece optical component are necessarily joined to each other, e.g., by portions of the optical component that extend around the light baffles. By having the various light guide components of the optical component as one piece, rather than e.g. separately connected to the housing or other part of the device, for example, registration of the light guide components relative to each other is entirely defined during forming of the optical component.

According to one aspect of the present disclosure, there is provided a one-piece optical component for an assay device comprising:
a first excitation light guide;
a first emission light guide;
a second excitation light guide; and
a second emission light guide.

The first and second emission light guides may be configured as described above. For example, the first and second emission light guides may each include curved refractive surfaces at one or both ends of the light guides. At one or both ends of each light guide, a lens may be provided, e.g., a ball lens, half ball lens or a plano-convex lens. A spacer may be provided between the lenses. Convex surfaces of the lenses may project from ends surfaces of the spacer. The spacer may be a cylindrical spacer. The spacer may provide a total-internal-reflection concentrator between the two lenses. The first and second emission light guides may be relatively close to each other or even touching. The first and second emission light guides may have central axes that extend at different angles.

The first and second excitation light may be configured as described above. For example, the excitation light guides may each comprise a light collimator lens adapted to locate adjacent the respective light source, in order to collimate light arriving from the light source into the excitation light guide. The excitation light guides may also comprise refractive and/or reflective surfaces. The excitation light guides may comprise a light exit face. At least one refractive and/or reflective surface may be provided at a substantially opposite side of each light guide to the light exit face. The at least one refractive and/or reflective surface may be curved. A best fit plane extending through the curved surface may be at an angle from the plane of the light exit face. The angle may be between about 20 and 70 degrees, or between 30 and 50 degrees or otherwise. The curved surface may extend substantially an entire length of the light guide. The refractive and/or reflective surfaces may act as a combined folding mirror and lens, providing optical power to the excitation light.

In one embodiment, in a first direction of the assay device and/or optical component, the first and second excitation light guides are provided at substantially opposite ends of the optical component and the first and second emission light guides are located between the first and second excitation light guides. A first opening may be located in the optical component between the first excitation light guide and the first emission light guide and a second opening may be located in the optical component between the second excitation light guide and the second emission light guide. The first opening may be adapted to receive at least a portion of the first light baffle and the second opening may be adapted to receive at least a portion of the second light baffle.

The optical component may include an upper wall, including an upper surface that is adapted to face the one or more light sources and detectors. The emission and excitation light guides may each be at least partly integrated into the upper wall and may have portions that project upwardly and/or downwardly from the upper wall. The first and second openings may be located in the upper wall.

The optical component may include one or more side walls that extend from the upper wall, e.g. from side edges of the upper wall. The one or more side walls may extend downwardly from the upper wall. The one or more side walls may comprise first and second side walls that are located opposite to each other in a second direction of the assay device and/or optical component, the second direction being perpendicular to the first direction. The one or more side walls may extend, for example, along substantially the full length of the optical component in the first direction.

The one or more of the side walls may each comprise a first clip element. The first clip element may be located centrally, for example, in the respective side wall, in the first direction of the optical component. The first clip element may be a deformable portion of the respective side wall that is arranged to deform to engage a corresponding, second clip element of the housing, to secure the optical component to the housing. The one or more side walls may each comprise a cavity in which the first clip element is movable as it deforms to engage the second clip element. For example, the first clip element may flex inwardly into the respective cavity as the first clip element deforms to engage the second clip element. The cavity may be defined by a recessed section of the respective side wall. The recessed section of one of the side walls may be connected to a recessed section of the other of the side walls by a rib. The rib may serve to strengthen the recessed sections (which may be relatively thinner sections of the side walls or otherwise) and/or provide a platform to support or otherwise engage the test portion, e.g. lateral flow test strip. The rib may be located between the first and second emission light guides in the first direction of the optical component.

When the optical component is formed through injection moulding, the optical component may be formed such that a weld line, formed by separate fronts in the fluidic injection moulding material meeting during the injecting moulding process, is not situated within any one of the excitation or emission light guides. This may ensure that any weld line does not disrupt the optical path through the excitation or emission light guides. For example, the weld line may be located on one of the side walls.

To control the location of the weld line, the optical component may comprise an injection flow control region. The injection flow control region may correspond to a narrowing of the injection mould that may provide a ‘bottleneck’ to flow of the injection moulding material in the injection mould, the injection flow control region being characterised by a corresponding narrowing that is formed in the optical component. The injection flow control region may be present in one of the side walls but not the other of the side walls. By providing a ‘bottleneck’ to flow of the injection moulding material, a front of fluidic injection moulding material moving through the mould region corresponding to one side wall may move slower than a front moving through the mould region corresponding to the opposite side wall. This may ensure asymmetry of injection material flow and therefore movement of the weld line away from a centrally positioned optical path within the optical component.

### BRIEF DESCRIPTION OF DRAWINGS

By way of example only, embodiments are now described with reference to the accompanying drawings, in which:

FIG. 1 shows a cross-sectional view of a portion of an assay device according to an embodiment of the present disclosure;

FIG. 2 shows a close-up of the cross-sectional view of FIG. 1 generally at a region indicated by reference A in FIG. 1;

FIG. 3 shows a ray trace diagram for optical components of the device of FIG. 1;

FIG. 4 shows an oblique view of a housing base employed in the device of FIG. 1;

FIG. 5a shows an oblique view of a test strip used in the assay device of FIG. 1; and FIG. 5b provides a schematic representation of the test strip including several regions that are arranged sequentially along the length of the strip of FIG. 5b;

FIGS. 6a and 6b show top and bottom oblique views, respectively, of an optical component according to an embodiment of the present disclosure employed in the device of FIG. 1; and

FIG. 7 shows plots of excitation and emission spectra of quantum dots that may be used in embodiments of the present disclosure.

### DESCRIPTION OF EMBODIMENTS

FIG. 1 shows a cross-sectional view of a portion of an assay device 1 according to a first embodiment of the present disclosure. The device 1 comprises a housing 10 including an outer casing 11 and an elongate base 12 located within the outer casing 11. The housing 10 defines an interior region in which a test portion, electronic circuitry and an optical component 40 are located.

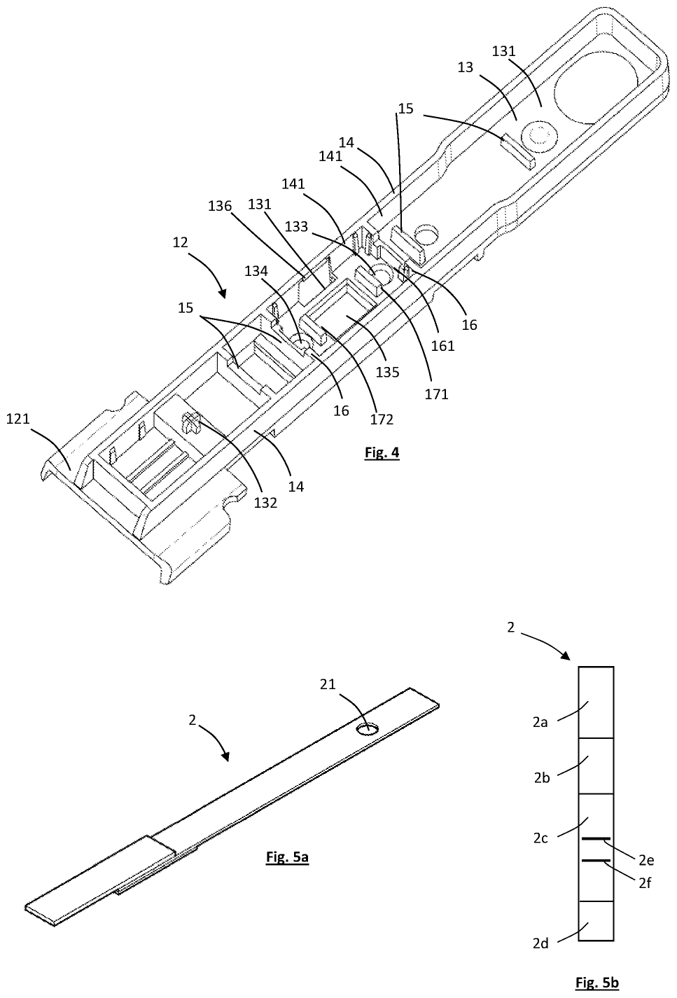

The base 12 of the housing 10 is shown in more detail in FIG. 4. The base 12 includes a rear wall 13 and side walls 14 projecting from the edges of an inner surface 131 of the rear wall 13, the inner surface 131 of the rear wall and inner surfaces 141 of the side walls 14 defining a recessed portion of the base 12. The recessed portion is substantially enclosed on one side by the test portion, more particularly, in this embodiment, a lateral flow test strip 2, as represented in FIG. 5a. Adjacent a first end 121 of the housing, a protuberance 132 projects in a direction away from the inner surface 131 of the rear wall 13 and is adapted to fit into a registration hole 21 of the test strip 2. In combination with guide reliefs and ribs 15 formed in the housing base 12, the engagement between the protuberance 132 and the hole 21 in the test strip 2 serves to register the position of the test strip 2 relative to the base 12 and other components of the assay device 1.

As indicated, the test portion in this embodiment is provided by a lateral flow test strip 2. The lateral flow test strip 2 includes several regions that are arranged sequentially along the length of the strip, as represented schematically in FIG. 5b. The regions include a sample receiving region 2a, a label-holding region 2b (although an alternative approach to using the label-holding region 2b is discussed below), a test region 2c, and a sink 2d. The regions comprise chemically treated material such as chemically treated nitrocellulose, located on a waterproof layer. The design of the test strip 2 is such that a biological sample, when received and transferred from the sample receiving region 2a can transfer under capillary action into the label-holding region 2b, which contains a fluorescent substance for labelling target analytes in the sample, and into the test region 2c where the sample will contact first and second test zones (first and second test stripes 2e, 2f in this embodiment) which each contain an immobilized compound capable of specifically binding the target analytes or a complex that the target analytes and the fluorescent labelling substance form. The sink (absorbent) region 2d is provided to capture any excess sample. Transfer of the sample along the test strip 2 can be assisted using a buffer solution, e.g., a buffer solution released from a reservoir in the device 1 or that has been combined directly with the biological sample before receipt of the sample at the sample receiving region 2a. The presence of the fluorescent labelled analyte in the sample generally results in at least one of the test stripes 2e, 2f at the test region 2c being excitable by light in a particular wavelength band such as to cause a detectable level of florescent emission light (fluorescent signal) to be emitted by the fluorescent label in a different wavelength band. Depending on the degree of light or signal detected, for example, it may be determined that the target analyte is present in the sample, and therefore a person providing the sample has a particular medical condition.

In this embodiment, the assay device 1 is configured to test for the presence of both influenza A and influenza B analytes in a biological sample. The first test stripe 2e is configured to bind fluorescent-labelled influenza A analyte/complex, if the influenza A analyte is present in the sample, and the second test stripe 2f is configured to bind fluorescent-labelled influenza B analyte/complex, if the influenza B analyte is present in the sample. Although not shown, a further control stripe may also be provided to indicate that a testing procedure has been performed. The control stripe can be located downstream of the first and second test stripes 2e, 2f to bind and retain the labelling substance. Detection of fluorescence at the control stripe can indicate that sample has flowed through the test region 2c.

In alternative embodiments, e.g., where a single target analyte is to be detected only, e.g., influenza A only, influenza B only, or an entirely different target analyte, the second test stripe 2f may be provided for use as a control stripe only. Nonetheless, where target analytes such as influenza A and influenza B are to be detected, in accordance with the present embodiment, since the presence of one of these analytes in a body is generally understood to be mutually exclusive of the presence of the other of these analytes in the body, one of the two test stripes 2e, 2f can be used to perform a control function. For example, background fluorescence or autofluorescence can be deduced from the test stripe that does not have a fluorescently labelled target analyte bound thereto, and the value of this fluorescence can be considered when determining a degree of fluorescence attributable to the presence of fluorescently labelled target analyte at the other of the test stripes 2e, 2f.

In an alternative embodiment, rather than using the label-holding region 2b on the test strip 2 for the labelling of the biological sample, labelling is carried out separately from the test strip 2.

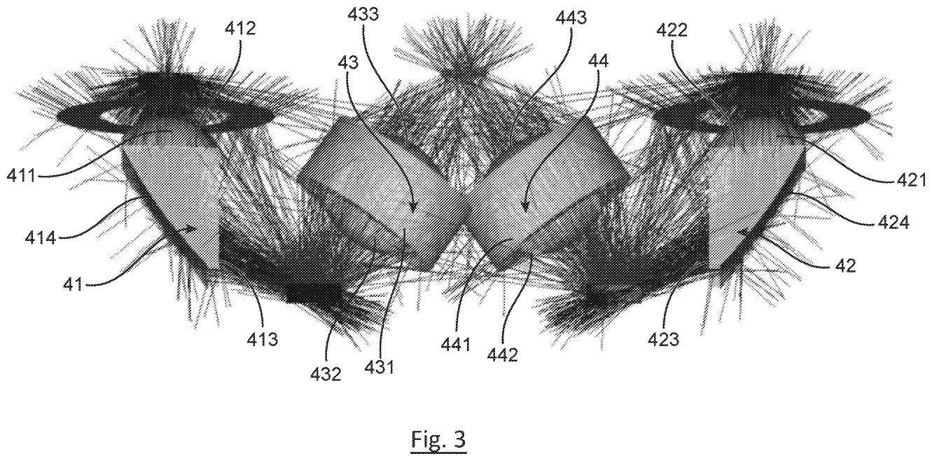

Prior to receipt at the sample receiving region 2a, the labelling may be carried out, through an incubation process, as exemplified, for example, in PCT publication no. WO 2019/006500A1 (with reference to FIGS. 3a to 3c). The entire content of PCT publication no. WO 2019/006500A1 is incorporated herein by reference. After the incubating, the sample may be applied to the sample receiving region 2a such that the sample, including any labelled complexes, flows from the receiving region 2a to at least the first and second test stripes 2e, 2f of the test strip 2.

By labelling the sample through an incubation process, labelled analytes/complexes may be distributed relatively uniformly throughout the sample. A relatively homogenous labelled sample may therefore be received at the sample receiving region 2a and then the first and second test stripes 2e, 2f. This may provide, during the assay period, a substantially linear increase over time of the signal at one of the test stripes 2e, 2f and a substantially constant signal at the other one of the test stripes 2e, 2f, for example.

Thus, the assay device 1 may make a determination about the first target analyte by monitoring a change in levels of first and second signals (fluorescent light signals) at the first and second test stripes 2e, 2f, respectively, over an assay period. If the first analyte (e.g. influenza A) of interest is present in the sample, the level of e.g. the first signal can increase during the assay period, while the second signal remains constant. The assay device 1 may also make a determination about the second target analyte in the sample. If the second analyte of interest (e.g. influenza B) is present in the sample, the level of e.g. the second signal can increase during the assay period, while the first signal remains constant. The approach is discussed further in PCT publication no. WO 2019/006500A1, the entire content of which being incorporated herein by reference.

By making determinations about the first and/or second analytes based on monitoring of a change between first and second signal levels over an assay period, more accurate determinations to be made about the first and/or second analytes in the sample, without being affected by ‘noise’ caused by autofluorescence or undersirable light transfer through the optical component. The approach may reduce any need to carry out spectral filtering within the excitation and emission light guides, and may support use of a one-piece optical component comprising the excitation and emission light guides and/or use of a spectral filter associated with the light detector as discussed herein.

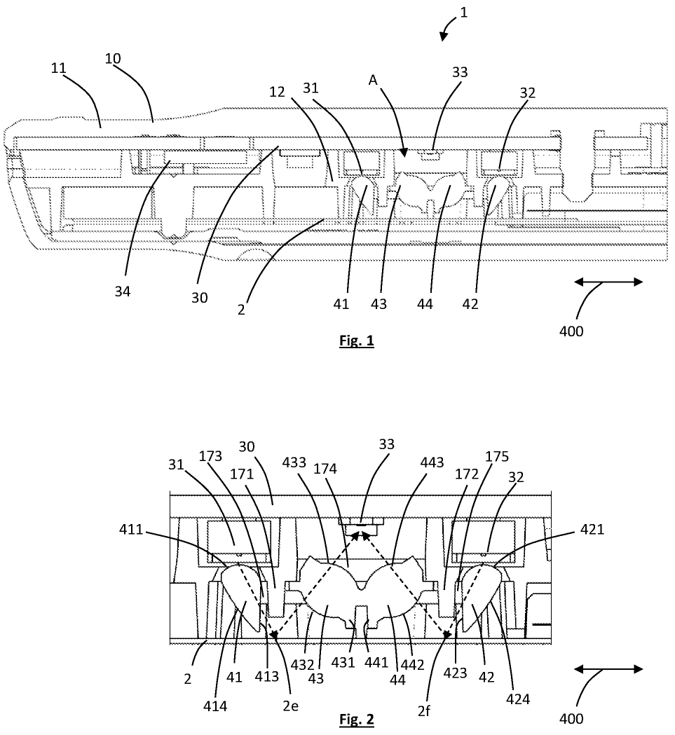

Referring to FIGS. 1 and 2, the electronic circuitry includes a first light source, in particular a first LED 31, a second light source, in particular a second LED 32, and a light detector, in particular a photodetector 33. The photodetector 33 is positioned between the first and second LEDs 31, 32. The first LED 31 is adapted to illuminate the first test stripe 2e and the second LED 32 is adapted to illuminate the second test stripe 2f. The electronic circuitry includes a printed circuit board 30 connected to a battery and processing chip/processor 34. The first and second LEDS 31, 32 and the photodetector 33 are located on and protrude from the circuit board 30. The circuit board 30 is positioned adjacent an opposite side of the rear wall 13 of the base 12 of the housing 1 from the test strip 2, between the base 12 and a portion of the casing 11. With reference to FIG. 4, access holes 133, 134, 135 are provided in the rear wall 13 of the housing to allow light to transfer from the LEDS 31, 32 to the optical component 40, and from the optical component 40 to the photodetector 33. The access holes include first and second LED access windows 133, 134 and a photodetector access window 135 positioned between the first and second LED access windows 133, 134.

The wavelength of light of the first and second LEDs 31, 32 is chosen so as to excite any fluorescent-labelled analytes that are bound at either of the first and second test stripes 2e, 2f, and therefore cause emission of fluorescent light from the test stripes 2e, 2f. The photodetector 33 is configured to detect the fluorescent emission light and, based on the strength of light detected, the processor of the device is adapted to make a determination about the presence of influenza A or influenza B in the biological sample.

In this embodiment, the photodetector 33 is effectively shared between the two LEDs 31, 32 and the two test stripes 2e, 2f. In order to differentiate between emission light from the first test stripe 2e and emission light from the second test stripe 2f, the LEDs 31, 32 may be adapted to illuminate the respective test stripes 2e, 2f at different times, e.g., sequentially. However, separate photodetectors 33 may be used in alternative embodiments.

Referring to FIGS. 1, 2 and 4, the optical component 40 is generally disposed between the electronic circuitry including the printed circuit board 30 and the lateral flow test strip 2, in a central recess of the base 12 defined between the inner surface 131 of the rear wall 13 of the base 12, the inner surfaces 141 of the side walls 14 of the base 12 and the inner surfaces 161 of two opposing inner walls 16 of the base 12, which are spaced apart in the elongate direction of the base 12.

Referring to FIG. 2, the optical component 40 includes first and second excitation light guides 41, 42 and first and second emission light guides 43, 44. The first excitation light guide 41 is adapted to guide excitation light from the first LED 31 to the first test stripe 2e and the second excitation light guide 42 is adapted to guide excitation light from the second LED 32 to the second test stripe 2f. The first emission light guide 43 is adapted to guide fluorescent emission light from the first test stripe 2e to the photodetector 33 and the second emission light guide 44 is adapted to guide fluorescent emission light from the second test stripe 2f to the photodetector 33. Path directions of the light are represented very generally using dotted arrows in FIG. 2. FIG. 2 shows a close up view of the device 1 at a region indicated by reference A in FIG. 1. A ray-trace diagram, shown in FIG. 3, provides a more detailed representation of excitation and emission light travelling through the optical component 40.

The excitation and emission light guides 41, 42, 43, 44 perform multiple functions. For example, in addition to guiding light to and from the test stripes 2e, 2f, the light guides focus, collimate and/or diverge light.

In this embodiment, however, the excitation and emission light guide 41, 42, 43, 44 are configured such that they perform substantially no spectral filtering of light.

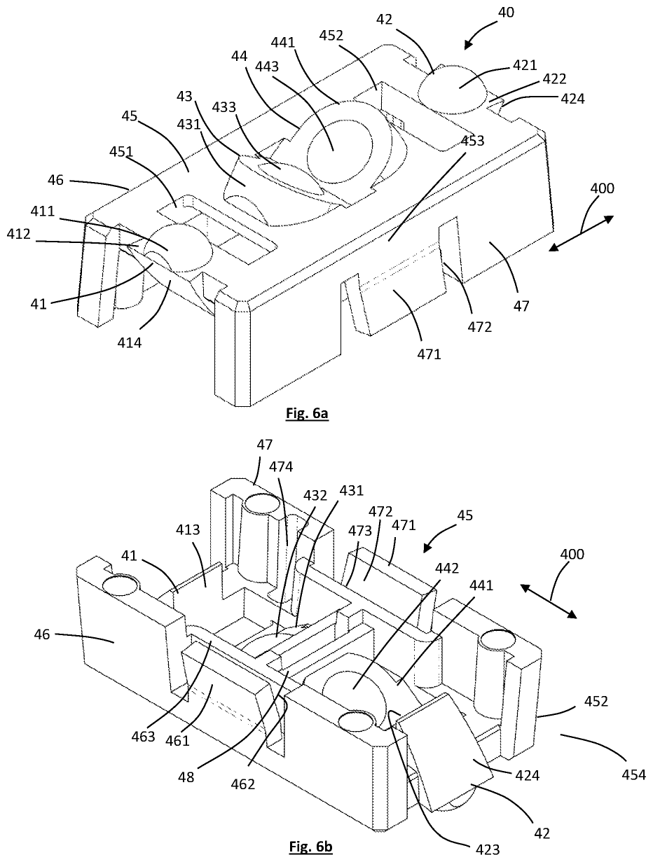

With reference to FIGS. 2, 6a and 6b, each excitation light guide 41, 42 includes a convex light collimator lens 411, 421 positioned on a first end face 412, 422 of the light guide adjacent the respective light source 31, 32. The collimator lenses 411, 421 are configured to collimate light arriving from the respective light source 31, 32 into the excitation light guide 41, 42. Each excitation light guide 41, 42 also includes a light exit face 413, 423 and a light focusing reflector face 414, 424, which each extend towards the test strip 2 from opposite edges of the first end face 412, 422. The light exit face 413, 423 extends substantially perpendicular to the test strip 2, whereas the reflector face 414, 424 extends at an acute angle relative to the test strip 2. As the light exit face 413, 423 and the reflector face 414, 424 extend from the first end face 414, 424, they converge together, giving the excitation light guides 41, 42 a substantially wedge shaped configuration. A best fit plane extending through the reflector surface 414, 424 is at an angle from the plane of the light exit face of about 30 to 50 degrees, e.g., about 40 degrees. In general, the first and second excitation light guides 41, 42 are configured to route excitation light efficiently from the first and second LEDs 31, 32 to the first and second test stripes 2e, 2f, respectively. The reflector surface 414, 424 provides a combined mirror and lens in this embodiment, giving optical power to the excitation light as it passes through the excitation light guides 41, 42.

Each emission light guide 43, 44 includes a body 431, 441 (or ‘spacer’) with first and second end opposite surfaces. The first and second end surfaces are provided with first and second convex lenses 432, 433, 442, 443, respectively, which lenses are adapted to focus light into and out of the body 431, 441. The convex lenses may be ball lenses, half ball lenses or plano-convex lenses). While most light may be transferred directly between the two convex lenses 432, 433, 442, 443 of each emission light guide 43, 44, some light may also be routed by total internal reflection (TIR) within the cylindrical body 431, 441.

With reference to FIGS. 6a and 6b, the optical component 40 is a one-piece component and therefore the first and second excitation light guides 41, 42 and the first and second emission light guides 43, 44 are formed together as one-piece. The optical component 40 in this embodiment is one-piece by being moulded, and particularly injection-moulded, as a single piece of homogenous material. The optical component 40 is generally monolithic, solid and unbroken. In this embodiment, the optical component is formed of a clear plastic material.

In a first direction 400 of the assay device and/or optical component, the first and second excitation light guides 41, 42 are provided at substantially opposite ends of the optical component 400 and the first and second emission light guides 43, 44 are located between the first and second excitation light guides 41, 42.

The optical component 40 includes an upper wall 45, including an upper surface 451 that is adapted to face generally towards the LEDs 31, 32 and photodetector 33. The emission and excitation light guides 41, 42, 43, 44 are each partly integrated into the upper wall 45 and have portions that project upwardly and downwardly from the upper wall 45.

The optical component 40 include two opposing side walls 46, 47 that extend downwardly from the upper wall 45. The side walls 46, 47 are located opposite to each other in a second direction that is perpendicular to the first direction 400. The side walls 46, 47 extend along substantially the full length of the optical component 40 in the first direction 400.

A first opening 451 is located in the optical component 40 between the first excitation light guide 41 and the first emission light guide 43 and a second opening 452 is located in the optical component 400 between the second excitation light guide 42 and the second emission light guide 44. The first and second openings 451, 452 are located in the upper wall 45 in this embodiment.

In this embodiment, the side walls 46, 47 each comprise a first clip element 461, 471. The first clip element 461, 471 is located centrally in the respective side wall 46, 47, in the first direction 400 of the optical component 40. The first clip element 461, 471 is provided by a deformable portion of the respective side wall 46, 47 and is arranged to deform to engage a corresponding, second clip element 136 (see FIG. 4) of the housing 10 and more particularly the base 12 of the housing 10, to secure the optical component 40 to the housing 10. The side walls 46, 47 each comprise a cavity 462, 472 in which the first clip element 461, 471 is movable as it deforms to engage the second clip element 136. The first clip element 461, 471 can flex inwardly into the respective cavity 462, 472 as the first clip element 461, 471 deforms to engage the second clip element 136. The cavity 462, 472 is defined by a recessed section 463, 473 of the respective side wall 46, 47. The recessed section 463 of one of the side walls 46 is connected to the recessed section 473 of the other of the side walls 47 by at least one rib 48. The rib 48 strengthens the recessed sections 463, 473 (which are relatively thinner sections of the side walls 46, 47 in this embodiment) and also provides a platform to support or otherwise engage the test strip 2. The rib 48 is located between the first and second emission light guides 43, 44 in the first direction 400 of the optical component 40.

In this embodiment, the optical component 40 is formed through injection moulding. Typically, the injection moulding process leads to forming of a weld line (not shown) in the optical component 40, caused by separate fronts in the fluidic injection moulding material meeting during the injecting moulding process. The optical component 40 is formed such that the weld line is not situated within any one of the excitation or emission light guides 41, 42, 43, 44 and therefore does not disrupt the optical path through the excitation or emission light guides 41, 42, 43, 44. For example, the weld line may be located in one of the side walls 46, 47.

To control the location of the weld line, the optical component 40 includes an injection flow control region 474. The injection flow control region 474 corresponds to a narrowing of the injection mould that provides a ‘bottleneck’ to flow of the injection moulding material in the injection mould. The injection flow control region 474 is characterised by a corresponding narrowing being formed in the optical component 40. In this embodiment, the injection flow control region 474 is formed in one of the side walls 47 but not the other of the side walls 46. By providing a ‘bottleneck’ to flow of the injection moulding material, a front of fluidic injection moulding material moving through the mould region corresponding to one side wall 47 may move slower than a front moving through the mould region corresponding to the opposite side wall 46. This can ensure asymmetry of injection material flow and therefore movement of the weld line away from a centrally positioned optical path within the optical component 40.

Referring to FIGS. 2 and 4, the base 12 of the housing includes first and second light baffles 171, 172, which project from the inner surface 131 of the rear wall 13 of the base 12. The baffles 171, 172 are provided between the LEDs 31, 32 and the photodetector 33, to prevent or reduce direct light transfer between the LEDs 31, 32 and the photodetector 33. The first and second light baffles extend through the first and second openings 451, 452, respectively, of the optical component 40. Distal ends of the first and second light baffles 171, 172 terminate short of the lateral flow test strip 2, substantially in line with the first and second test stripes 2e, 2f, to create openings between chambers that are defined between the light baffles 171, 172.

In more detail, the light baffles 171, 172 divide the central recess of the base 12 of the housing 1 into first, second and third chambers 173, 174, 175. The first chamber 173 and the second chamber 174 are separated by the first light baffle 171, and the second chamber 174 and the third chamber 175 are separated by the second light baffle 172. The first LED access window 133 opens into the first chamber 173, the photodetector access window 135 opens into the second chamber 174, and the second LED access 134 opens into the third chamber 175. The first test stripe 2e is positioned at the opening between the first chamber 173 and the second chamber 174, and the second test stripe 2f is positioned at the opening between the second chamber 174 and the third chamber 175. The first excitation light guide 41 is positioned in the first chamber 173, the second excitation light guide is positioned in the third chamber 175, and the first and second emission light guides 43, 44 are both positioned in the second chamber 174, between the first and third chambers 173, 175.

In this embodiment, the device 1 provides a rapid flu test (RFT), including a relatively low cost, inherently disposable, and high performance optical reader for a test strip 2. The device can digitise a pair of fluorescently labelled test stripes 2e, 2f and also a control stripe (via a subsidiary sensor arrangement not shown). The device employs an immunochromatographic (lateral flow) test strip 2 but can be adapted to alternative formats.

A function of the light guides 41, 42, 43, 44 is to provide efficient routing of light from the LEDs 31, 32 to the test strip 2 and from the test strip 2 to the photodetector 33. The light guides 41, 42, 43, 44 take a very compact, thin form, compatible with volume and low cost manufacture.

In order to route the light efficiently, the light guides 41, 42, 43, 44 in this embodiment use a combination of refractive surfaces and reflective surfaces. By employing refractive surfaces, e.g., in the emission light guides 43, 44, for example, total internal reflection (TIR) can be used, obviating the need for, and associated cost of, metallised (or equivalent) high reflectivity coatings.

In the assay device 1 of the present embodiment, the choice of fluorescent label is made in view of a range of considerations, including: the excitation wavelength band of the label (which affects the choice of light source and its associated power and cost); the absorptivity of the label, the efficiency of the label, e.g., quantum efficiency, of the label; the emission wavelength band of the label, (which affects the choice and cost of filters to separate the emission light from the excitation light), and the assay integration compatibility.

The fluorescent reader assembly described herein relies on being able to separate relatively strong excitation light from relatively weak fluorescent emission light, by virtue of their different wavelengths.

While traditional fluorescent labels may be used in embodiments of the present disclosure, they can exhibit relatively small Stokes shifts (i.e. a relatively small wavelength shift between the emission and excitation wavelength bands) and this places strong demands on filters, which can in turn translate to more expensive parts. It can also place stringent demands on all materials in the system to avoid contamination of the emission wavelength band by auto-fluorescence which typically exhibits small Stokes shifts.

It has therefore been found highly advantageous in embodiments of the present disclosure to operate the device with fluorescence based on larger (effective) Stokes shifts. Fluorophores that have been found to exhibit particularly large Stokes shifts are quantum dots, which are semiconductor nanoparticles, specifically engineered to create a particular excitation and emission response. Collectively, quantum dots can also offer relatively high absorption in the ‘short blue’ spectrum (which is conveniently accessible to low cost LEDs) and provides high quantum efficiency. Therefore, quantum dots have been determined as an appropriate fluorescent label according to the present disclosure, albeit other types of fluorescent labels could be used.

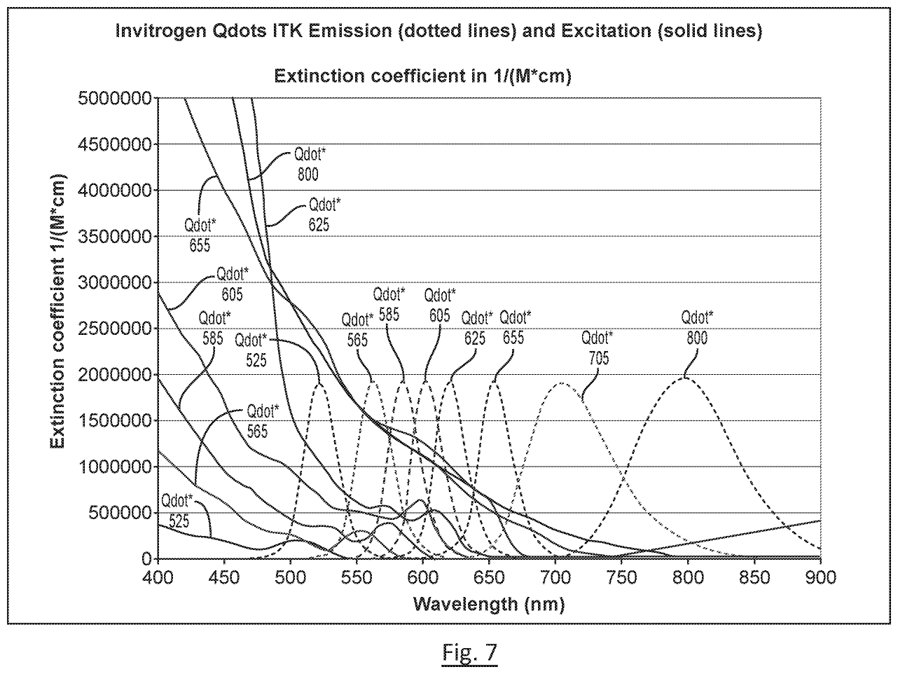

The label that is used in the assay device according to the present embodiment is a quantum dot with an emission wavelength band peak of 800 nm. An example of a quantum dot with such an emission wavelength band peak is the Invitrogen™ Qdot™ 800. Referring to FIG. 7, which shows quantum dot excitation and emission spectra, the Qdot™ 800 exhibits a relatively long Stokes shift compared to other quantum dots tested. This relatively high degree of shift simplifies filter choice and reduces auto-fluorescent contamination. Nonetheless, quantum dots with different excitation and emission spectra, e.g., as per any one of the quantum dots identified in FIG. 7, may be employed in embodiments of the present disclosure.

In the assay device of the present embodiment, the choice of light source, e.g., LED, has been made in view of a range of considerations, including: the absorptivity of the selected fluorescent label at the light source's operating wavelength; total radiant power at the maximum available drive current for the device; the available area of the electronic circuitry, and the device cost. When using the Qdot™ 800, absorptivity of about 350 nm is favoured, for example. However, quantum dots with an excitation wavelength band centred above or below 350 nm may be used, e.g., between 325 to 500 nm, between 350 nm to 450 nm, or centred around 405 nm, or otherwise. LEDs of higher wavelength can have lower manufacturing costs, and, while the higher wavelengths may not be most optimally absorbed by the fluorescent label, the may still be sufficiently absorbed by the fluorescent label for the purposes of the present disclosure.

LEDs selected for use in the assay device according to the present embodiment are surface mounted LEDs that typically emit at 405 nm. They provide high radiant power at a 30 mA operating current and compatibility with other components of the device. Nevertheless, as indicated, LEDs or other light sources having a variety of different wavelengths can be used.

As discussed, high discrimination between the emission and excitation wavelength bands by the light guides is desirable. There can therefore be high acceptance of light in the emission wavelength band at the photodetector and high rejection of light in the excitation wavelength band at the photodetector (in addition to high rejection of any excitation component that could overlap with emission band at the light source). In the present embodiment, this is achieved without using light guides that provide spectral filtering and instead using a spectral filter that is associated or integrated with the photodetector 33. In particular, the photodetector 33, through its integral spectral filtering properties, can offer high transmission of light in the emission wavelength band whilst blocking shorter wavelength light (i.e. it acts as a long pass filter). For example, in one embodiment, the photodetector 33 can only pass/detect a wavelength between −700 nm to −1.1 mm, corresponding to the wavelength of infrared light emitted by the quantum-dot particles.

In the present embodiment, the test strip is the primary ‘transducer’ that converts the target analytes (influenza A and B, or more particularly, nucleoproteins for influenza A and B) to, in essence, a density of quantum dot labels at pre-defined capture stripes 2e, 2f. In a fixed optics reader, it is essential that tight registration is achieved such that the fixed reader is centred on the stripes. In the present embodiment, this is achieved by using the housing 1, including the base 12, as a mechanical hub. Registration of the optics to the signal stripes is achieved at least in part by the protruberance 132 and the registration hole 21 of the test strip 2 (a form of pin and hole interface). The same hole can be used during test strip manufacture to register the positions of the test stripes 2e, 2f, relative to the stripes dispensing equipment.

Fluorescence detection requires the ability to measure very low light levels. In the present embodiment, the photodetector is a light to frequency converter. Ultimately, the photodetector can generate an electrical signal indicative of the strength of the fluorescent emission light that it detects from each of the first and second test stripes 2e, 2f, which signal is received by a processor 34 in the device 1. The amount of the target analyte can be determined by the processor correlating the strength of the fluorescent emission light to a predetermined target analyte concentration. However, the strength of the fluorescent emission light of one test stripe can also be compared by the processor with the strength of the fluorescent emission light of the other test stripe and a change in the relative strengths may be monitored over an assay period to determine target analyte concentration or otherwise.

For example, one process that can be employed by the present embodiment, relies on the fact that influenza A and B are mutually exclusive, or are at least very rarely seen in combination. Thus it can be expected that the result of the assay device will either be: influenza A positive or influenza B positive or neither influenza A or B positive. On this basis, the process can comprise:
measuring optical intensity signals at the first and second test stripes (the A & B lines), when the test strip is still dry, e.g., when the sample/buffer solution has not developed along the strip;
monitoring optical intensity signals at the A & B lines during development (e.g., to check for correct operation and to judge when the test is complete);
measuring optical intensity signals at the A & B lines at completion of the development;
normalising the A & B optical intensity signals using the dry values where this assumes that any background is common to the two channels;
calculating the difference in the two signal intensities and comparing a change in magnitude of the difference over an assay period, whilst using the sign to distinguish between A or B being positive

The approach may provide for robust measurement and permit use of a lower threshold for greater sensitivity. The approach may enable a one-piece optical component to be used, which includes both excitation and emission light guides and does not include spectral filtering features.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. An assay device comprising:
a housing; and
a test portion, electronic circuitry and an optical component, each a least partially located in the housing; wherein
the test portion comprises one or more test zones adapted to receive an analyte and a fluorescent label associated with the analyte, the fluorescent label being excitable by excitation light and adapted to emit emission light upon excitation by excitation light;
the electronic circuitry comprises one or more light sources and one or more light detectors; and
the optical component is a one-piece component that comprises one or more excitation light guides adapted to guide excitation light from the one or more light sources to the one or more test zones, and one or more emission light guides adapted to guide emission light from the one or more test zones to the one or more light detectors.

2. The device of claim 1, wherein the excitation and/or emission light guides focus, collimate and/or diverge light.

3. The device of claim 1 or 2, wherein the excitation and emission light guides include no spectral filtering function.

4. The device of claim 1, 2, or 3, wherein the one-piece optical component is formed of clear plastic.

5. The device of any one of the preceding claims, comprising one or more spectral filters associated or integrated with the one or more light detectors.

6. The device of claim 5, wherein the one or more spectral filters comprise a long pass filter.

7. The device of any one of the preceding claims, wherein an excitation wavelength band of the excitation light and an emission wavelength band of the emission wavelength band have peak wavelengths that are at least 200 nm apart, or at least 250 nm apart, or at least 300 nm apart, or at least 350 nm apart, or at least 400 nm apart.

8. The device of any one of the preceding claims, wherein an excitation wavelength band of the excitation light has a peak wavelength between 325 and 500 nm.

9. The device of any one of the preceding claims, wherein an emission wavelength band of the emission light has a peak wavelength between 650 and 850 nm.

10. The device of claim 7, 8 or 9, wherein the excitation wavelength band has a peak wavelength of about 405 nm and the emission wavelength band has a peak wavelength of about 800 nm.

11. The device of any one of the preceding claims, wherein the fluorescent label is a quantum dot.

12. The device of claim 11, wherein the quantum dot has a peak emission wavelength that is greater than 700 nm or greater than 750 nm

13. The device of claim 12, wherein the quantum dot has a peak emission wavelength of about 800 nm.

14. The device of any one of the preceding claims, wherein the optical component is moulded as a single piece.

15. The device of any one of the preceding claims, wherein the optical component comprises an upper wall, including an upper surface that is adapted to face the one or more light sources and the one or more light detectors, the emission and excitation light guides each at least partly integrated into the upper wall.

16. The device of claim 15, wherein the emission and excitation light guides each have portions that project upwardly and/or downwardly from the upper wall.

17. The device of claim 15 or 16, wherein the optical component comprises one or more side walls that extend downwardly from the upper wall.

18. The device of claim 17, wherein the one or more side walls comprise first and second side walls.

19. The device of claim 17 or 18, wherein each of the one or more of the side walls comprises a first clip element, the first clip element being a deformable portion of the respective side wall that is arranged to deform to engage a corresponding, second clip element of the housing to secure the optical component to the housing.

20. The device of claim 19, wherein each of the one or more side walls comprises a cavity in which the first clip element is movable as it deforms to engage the second clip element.

21. The device of claim 20, wherein the cavity is defined by a recessed section of the respective side wall.

22. The device of claim 21, wherein the recessed section of one of the side walls is connected to a recessed section of another of the side walls by a rib.

23. The device of any one of the preceding claims, wherein the optical component is formed by injection moulding and the optical component comprises an injection flow control region that controls a location of a weld line in the optical component.

24. The device of claim 22, wherein the injection flow control region is a narrowing of the optical component.

25. The device of any one of the preceding claims, wherein the one or more test zones comprise at least first and second test zones, and the optical component comprises at least first and second excitation light guides and first and second emission light guides, wherein the first excitation light guide is adapted to guide light from the at least one light source to the first test zone and the first emission light guide is adapted to guide light from the first test zone to the at least one light detector, and the second excitation light guide is adapted to guide light from the at least one light source to the second test zone and the second emission light guide is adapted to guide light from the second test zone to the at least one light detector.

26. The device of claim 25, wherein the first test zone is adapted to receive an influenza A analyte and the second test zone is adapted to receive an influenza B analyte.

27. The device of claim 25 or 26, comprising at least first and second light sources, wherein the excitation light guided by the first excitation light guide is from the first light source and the excitation light guided by the second excitation light guide is from the second light source.

28. The device of claim 27, wherein the first and second emission light guides are adapted to guide emission light from the first and second test zones to the same light detector.

29. The device of claim 28, wherein the light detector is located between the first and second light sources.

30. The device of any one of the preceding claims, wherein the device is a hand-held device.

31. The device of any one of the preceding claims, wherein the test portion is fixed relative to the electronic circuit and the optical component prior to receipt of a biological sample.

32. The device of claim 25, comprising a processor and a non-transitory computer-readable memory medium, the non-transitory computer-readable memory medium comprising instructions that cause the processor to:
monitor levels of first and second signals at the first and second test zones over an assay period wherein, if the first analyte of interest is present in the sample, the first analyte is fluorescently labelled and wherein the presence of the fluorescently labelled first analyte in the sample causes the level of one of the first and second signals to increase during the assay period; and
monitor a change between the first and second signal levels over a period of time during the assay period.

33. A one-piece optical component for an assay device, the one-piece optical component comprising:
a first excitation light guide;
a first emission light guide;
a second excitation light guide; and
a second emission light guide.
